(19) United States
(12) Patent Application Publication
(10) Pub. No.: US 2022/0039780 A1
(43) Pub. Date: Feb. 10, 2022

(54) Gum Graft Collector

(71) Applicant: Ricardo Kern, Curitiba - PR (BR)

(72) Inventor: Ricardo Kern, Curitiba - PR (BR)

(21) Appl. No.: 17/015,320

(22) Filed: Sep. 9, 2020

(30) Foreign Application Priority Data
Aug. 5, 2020 (BR) BR 202020015974-2

(51) Int. Cl. A61B 10/02; A61B 17/32

(52) U.S. Cl. CPC A61B 10/0266; A61B 2017/0042; A61B 17/32

(57) ABSTRACT

A graft collector designed to facilitate and offer greater precision in performing the cutting and “dissection” of an area of the donor tissue in grafting operations. The graft collector is set as an anatomical grip handle based on a support head and fixation of a removable curved or angled blade. The cutting blade is provided with a cutting edge on two opposite sides, being folded in two points at 90°. The constructive arrangement of the graft collector allows the fixation of the cutting blade and its loosening and release for replacement. The said graft collector is a precise and anatomical grip instrument, designed for the good manual performance of the professional when cutting and removing the tissue to be used as a graft.

### BACKGROUND OF THE INVENTION

This descriptive report refers to a request for a utility model for an instrument for graft collection, replacing the scalpel and other straight blades, especially in the collection of gum graft.

The collector has a precise and anatomical handle, being designed for the good manual performance of the professional. Its cutting blade, together with the physical conformation of the instrument, allows the collection of the perfect portion of the graft in a single cut, without the need for hand-made incisions, and with the appropriate dimensions and proportions.

### STATE OF THE ART

There is a growing demand for cosmetic dentistry treatments, whose already wide range of procedures is increasing every day. Among them, the new techniques and materials for gum plastic surgery, such as the gum graft, are gaining prominence and proving to be indispensable, either in the construction of more harmonious smiles or in the correction of anatomical flaws. Gum graft treatment is a simple yet delicate dental procedure. Idealized to be performed in the autogenous technique, in which the grafted gum tissue comes from the patient himself, it can also use alternative biosynthetic materials, obtained from animal matrices, or totally synthetic.

Three different types of gum graft are known to technicians and implantology professionals that can be performed to cover the gum retraction. The most used gum grafting techniques are:

Connective tissue gum grafting: this is the most used gum graft technique to treat gum swelling and root exposure. The region most indicated as a gum graft donor is the palate. During the procedure, the most superficial layer of the palate's gum is lifted, allowing the removal of an underlying layer, called subepithelial connective tissue. Once the connective tissue is removed, it is positioned and stabilized through suture points over the exposed root area. After the gum graft is performed, the superficial layer of gum on the palate is repositioned and sutured, allowing the wound to be completely closed.

Free gum graft: similar to a connective tissue gum graft, the free gum graft also has the palate region as a donor area. However, there is the use of the most superficial layer of gum for root coverage. This gum graft technique is best indicated in cases of root covering, without gaining gum volume.

Pedicled gum graft: this gum graft procedure is best for small coverings, as the tissue covering the root comes from the gum of the adjacent tooth. Small incisions are made in the most superficial and epithelial portion of the gum, close to the area to be covered, and then a movement of rotation and displacement of the tissue is performed, covering the root exposure, thus completing the gum graft.

As examples of the foregoing, the following prior art document, which represents the state of the applied art:

Brazilian patent document PI 0800323-8, entitled “APPARATUS FOR SURGICAL TECHNIQUES OF FREE AND CONNECTIVE SUB-EPITHELIAL GRAFT”, which demands a device to increase, in area, gum grafts, used in periodontics and implantology. Grafts such as those used for root coverage, as well as for gaining keratinized mucosa. Said apparatus works from a rotating roller that touches a sliding table, and the gap that forms between the roller and the table has an adjustable dimension according to the desirable thickness of the tissue to be grafted.

Although simple, the gum graft is a technique considered difficult by professionals. Its difficulty is especially in the removal (cut) of the tissue that will be used as a graft since the thickness of the flap and the amount of keratinized mucosa are essential for the success of the technique.

Nowadays, the removal or tearing of donor tissue is manually carried out, using a scalpel or other appropriate cutting blade. Therefore, it is seen that the technique is almost handmade and requires an experienced precision on the part of the professional, who is still susceptible to errors. This difficulty is intensified when taking into account that, after delimiting the cut area with the instrument, incisions must be made perpendicular to the area with such thickness that it allows a delicate dissection, avoiding leaving the exposed bone as this leads to an extremely healing slow and painful.

In addition, if the removed tissue is thicker than intended, after healing the area is coarse in appearance, showing the difference in volume between the graft and the remaining gum.

Therefore, the professionals in the area lack equipment that offers greater precision and a smaller margin of errors in the removal or dissection of the “graft”, since this can only be done nowadays using a scalpel or other linear blades.

### Objective of the Invention

Aware of the lack of specific instruments for facilitated graft collection, specifically gum graft, the inventor comes to present a graft collector in order to solve the problems described in the state of the art.

In summary, the graft collector is formed by an anatomical handle cable, with smooth contours and precise handle, which incorporates a head where a U-folded blade is located, supported and fixed, removable, by a threadable assembly.

The collector has a modern and harmonic design, presenting itself as a differentiated and correct instrument for professionals in the area.

The invention has as main objectives:
Offer a differentiated instrument that allows the collection of gum graft in few and simplified movements.
Offer a graft collection instrument that dispenses with the artisanal work of professionals, reducing the chances of errors;
Offer an anatomical graft collection instrument that is easy to use;
Offer a more current alternative to the scalpel;
Offer a graft collection instrument with easy blade change.

### BRIEF DESCRIPTION OF THE DRAWINGS

Explained until then in a brief way, the invention is illustrated by the accompanying drawings.

FIG. 1—isometric view of the graft collector. Detail A shows the blade fastening components in an exploded state;

FIG. 2—side view of the graft collector. Detail B shows the partial view, of the head, in lateral median section;

FIG. 3—front view of the graft collector. Detail C shows the head in frontal median section;

FIG. 4—frontal sectional view, according to detail C, showing the introduction and fixation of the curved blade in the coupling terminal.

### DETAILED DESCRIPTION OF THE INVENTION

In accordance with the attached drawings, the gum graft collector the reason for this patent application, is an innovative provision applied in a cutting instrument to collect gum graft. The instrument is more precisely presented as a graft collector (1), designed in order to facilitate and offer greater precision in performing the cutting and “dissection” of an area of the donor tissue in grafting operations.

In general, the graft collector (1) is set as an anatomical grip handle based on a support head and fixation of a removable curved or angled blade.

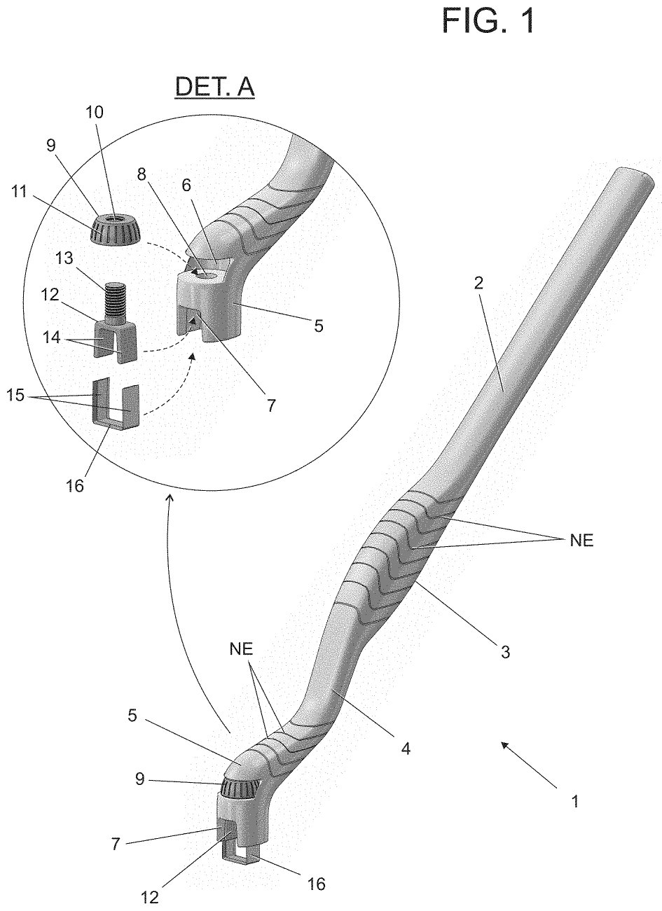

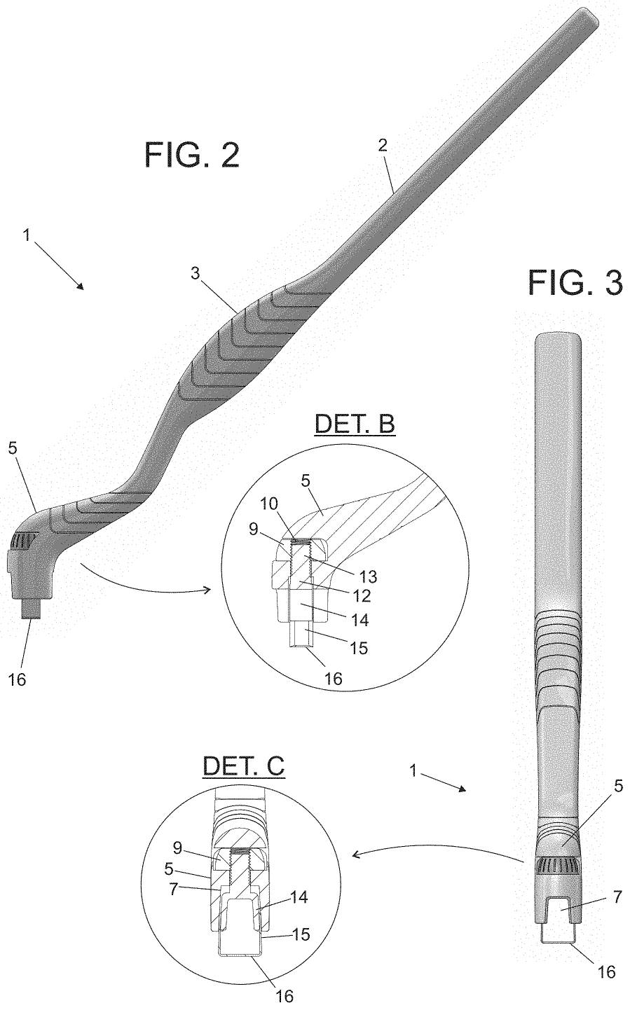

More specifically, as shown in FIGS. 1, 2 and 3, the graft collector's handle (1) is configured as an elongated body (2), anatomical, with an preferably oval profile, which extends to a belly of support (3) with sinuous contours, from which the body continues in a curvilinear neck (4) incorporating the head (5). The support belly (3) of the elongated body (2) and its union with the head (5) receive a pattern of reentrant ribs (NE) to aid the grip.

The said head (5) is rounded and extends downwards, being interrupted by a semi-circular housing (6) and continuing until a coupling terminal (7) formed by a through inverted U-slot. The semi-circular housing (6) and the coupling terminal (7) share a central hole (8).

The head (5) receives in its semi-circular housing (6) a fixing pulley (9) with a central threaded hole (10), and side ribs (11), being positioned in the housing (6) so that it can be rotated by user's finger with the aid of ribbings (11). The coupling terminal (7), in turn, receives a clamping component (12), formed from a straight base that extends superiorly a threaded neck (13), which is inserted in the central threaded hole (10) of the fixing pulley (9), and it extends inferiorly two opposing legs (14) and open at an angle in order to press against the walls of the terminal (7), the sides (15) of a U-folded cutting blade (16).

The cutting blade (16) is, more specifically, a rectangular blade segment with cutting edge on the two opposite sides, this segment being folded in two points at 90°, resulting in a three-part blade, equal or not, a horizontal base bounded by the vertical, folded sides.

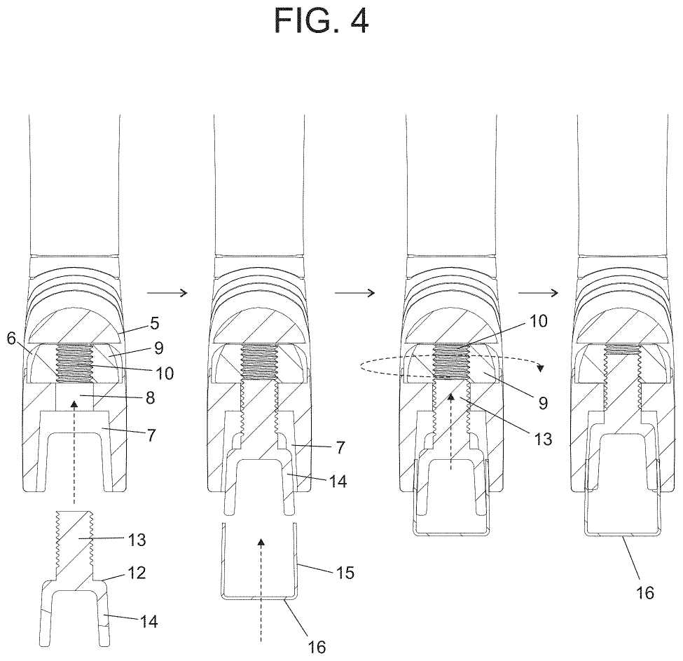

The constructive arrangement of the graft collector (1) allows the rotation of the fixing pulley (9) by the user in one direction to tighten the clamping component (12) against the terminal (7), where the legs (14) at an angle firmly fix the sides (16) of the cutting blade (16) by pressing against the walls of said terminal (7), as shown in FIG. 4, and turning the fastening pulley (9) in the opposite direction loosen the clamping component (12), releasing the blade (16) for its removal and replacement.

Thus, it is possible to identify the graft collector (1) as a precise and anatomical grip instrument, having been designed for the good manual performance of the professional when cutting and removing the tissue to be used as a graft. Its cutting blade (16), folded in a U shape, allows the simple slide through the donor tissue to cut this in a portion controlled by its horizontal part and bounded by the vertical folded parts, performing the collection of the perfect graft portion in a single cut, without the need to make incisions almost handmade. It is to say that the conformation of the blade (16) allows the graft collection to be carried out with the appropriate dimensions and proportions for its use. This cutting movement is further assisted and significantly facilitated by the conformation of the head (5), which provides the ideal support for the professional's finger to exert continuous and easily controlled pressure during the cut.

In addition, the blade (16) can be easily replaced by screwing and unscrewing the fixing pulley (9).

Finally, the general shape of the elongated body (2) of the graft collector (1) allows its perfect use in almost any position the patient is in offering the professional an excellent range with easy variation of the positioning angle, unlike a scalpel.

1. GUM GRAFT COLLECTOR (1) designed to perform the cutting and dissection of an area of donor tissue in graft operations, said graft collector (1) characterized by being configured as an anatomical handle found in a head of support and fixation of an angled, removable blade.

2. GUM GRAFT COLLECTOR, according to claim 1, characterized by the handle is an elongated body (2),
anatomical, with a preferably oval profile,
which extends to a support belly (3) with sinuous contours,
from which the body remains in curvilinear neck (4) incorporating the head (5).

3. GUM GRAFT COLLECTOR, according to claim 1, characterized by the support belly (3) and the union of the body (2) with the head (5) receive a pattern of reentrant ribbings (NE) to aid the grip.

4. GUM GRAFT COLLECTOR, according to claim 1, characterized by a rounded head (5), that extends downwards,
being interrupted by a semi-circular housing (6) and
continuing until a coupling terminal (7) formed by an inverted U-slot through, said semi-circular housing (6) and coupling terminal (7) sharing a central hole (8).

5. GUM GRAFT COLLECTOR, according to claim 1, characterized by head (5) receives in its semi-circular housing (6)
a fixing sheave (9)
with threaded central hole (10)
and side ribs (11),
positioned in the housing (6) in order to be rotated.

6. GUM GRAFT COLLECTOR, according to claim 1, characterized by coupling terminal (7) receiving a clamping component (12),
formed from a straight base that extends superiorly a threaded neck (13),
which is inserted into the central threaded hole (10) of the fixing pulley (9) and inferiorly extends two opposing legs (14)
and open at an angle in order to press against the walls of the terminal (7), the sides (15) of the U-folded cutting blade (16).

7. GUM GRAFT COLLECTOR, according to claim 1, characterized by the cutting blade (16) is a rectangular blade segment provided with cutting edge on the two opposite sides,
this segment being folded in two points at 90°, resulting in a three-part blade, equal or not, with a folded horizontal base bounded by the vertical sides.

8. GUM GRAFT COLLECTOR, according to claim 1, characterized by the physical arrangement of the graft collector (1) allowing the rotation of the fixing pulley (9) in one direction to tighten and fix the blade
(16) and the rotation of the fixing pulley (9) in the opposite direction loosen and release the blade (16).
